(19) United States
(12) Patent Application Publication
(10) Pub. No.: US 2009/0156952 A1
(43) Pub. Date: Jun. 18, 2009

(54) Apparatuses and Methods for Diagnosing and Treating Respiratory Conditions

(76) Inventors: C. Eric Hunter, Jefferson, NC (US); Lyndell Duvall, Fleetwood, NC (US); Philip Weaver, Mouth of Wilson, VA (US); Tom Stern, Charlotte, NC (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/972,265

(22) Filed: Jan. 10, 2008

Related U.S. Application Data
(60) Provisional application No. 61/013,438, filed on Dec. 13, 2007.

(51) Int. Cl. A61B 5/087; A61M 15/00

(52) U.S. Cl. 600/538; 128/203.12

(57) ABSTRACT

In one embodiment, a respiratory condition device includes a tube that defines a flow path for medicine that is to be delivered to a user respiratory system, a pressure sensor configured to detect pressure changes within the tube, and a medicine delivery device configured to eject medicine into the tube when a pressure drop is detected by the pressure sensor.

### CROSS-REFERENCE TO RELATED APPLICATION

This application claims priority to copending U.S. provisional application entitled, “Methods and Apparatus for Diagnosing and Treating Respiratory Conditions,” having Ser. No. 61/013,438, filed Dec. 13, 2007, which is entirely incorporated herein by reference.

### BACKGROUND

Asthma is a chronic disease of the respiratory system in which the airway occasionally constricts, becomes inflamed, and is lined with excessive amounts of mucus, often in response to one or more triggers. Such airway constriction causes symptoms such as wheezing, shortness of breath, chest tightness, and coughing.

The medical treatment recommended to patients with asthma depends on the severity of their illness and the frequency of their symptoms. Specific treatments for asthma include the administration of bronchodilators, which provide short-term relief to patients who experience an asthma attack. For those with mild persistent disease, low-dose inhaled glucocorticoids or alternatively, an oral leukotriene modifier, a mast-cell stabilizer, or theophylline may be administered. For those who suffer daily attacks, a higher dose of glucocorticoid in conjunction with a long-acting inhaled β-2 agonist may be prescribed. Alternatively, a leukotriene modifier or theophylline may substitute for the β-2 agonist. In severe asthmatics, oral glucocorticoids may be added to these treatments during severe attacks.

Symptomatic control of episodes of wheezing and shortness of breath is generally achieved with fast-acting bronchodilators, such as albuterol. These are typically provided in pocket-sized, metered-dose inhalers (MDIs). Typically, asthmatics self-administer bronchodilators or other drugs on an “as needed” basis. Therefore, no formal diagnosis is performed and the determination as to when to dose is left to the subjective discretion of the patient or the patient's guardian.

The above-described administration scheme is undesirable for various reasons. First, the asthmatic may not realize that medication is required until after an attack, possibly a severe attack, occurs. If the conditions that cause an attack could be identified earlier, such an attack could be avoided or its severity could be decreased. Second, the asthmatic may decide dosing is necessary even when it in fact is not. Such unnecessary administration of drugs is undesirable because overuse of the drugs may cause their efficacy to decline.

In view of the above, it would be desirable to be able to more accurately diagnose a respiratory condition before administering drugs to treat the condition.

### BRIEF DESCRIPTION OF THE DRAWINGS

The disclosed methods and apparatus can be better understood with reference to the following drawings. The components in the drawings are not necessarily to scale.

FIG. 1A is a top perspective view of an embodiment of a device for diagnosing and treating a respiratory condition.

FIG. 1B is a bottom perspective view of an embodiment of the device of FIG. 1A.

FIG. 2 is a schematic diagram of an embodiment of the device of FIGS. 1A and 1B.

FIG. 3 is a block diagram of an embodiment of a controller shown in FIG. 2.

FIGS. 4A-4C comprise a flow diagram that illustrates an embodiment of a method for diagnosing a respiratory condition.

FIG. 5 is a flow diagram that illustrates an embodiment of a method for treating a respiratory condition.

FIG. 6 is a schematic view of an embodiment of a system for diagnosing and treating a respiratory condition.

FIG. 7 is a schematic diagram of an embodiment of a diagnosis/treatment device shown in FIG. 6.

FIGS. 8A-8C comprise a flow diagram that illustrates an embodiment of operation of the system of FIG. 6.

FIG. 9 is a flow diagram that illustrates an embodiment of a method for remotely adjusting medication dosage.

FIGS. 10A-10B illustrate alternative embodiments of systems for diagnosing and treating a respiratory condition.

FIG. 11 is a front view of a further embodiment of a device for diagnosing and treating a respiratory condition.

### DETAILED DESCRIPTION

As described above, it would be desirable to be able to diagnose a respiratory condition, such as an asthma attack, before administering medication. As described in the following, such diagnosis can be performed using a device that measures relevant respiratory system parameters and automatically determines whether such a condition exists. In some embodiments, the medication can then be administered with the device.

In the following, various embodiments of methods and apparatus are described. It is to be understood that those embodiments comprise mere implementations of the disclosed inventions and that alternative embodiments are possible and are intended to fall within the scope of the present disclosure.

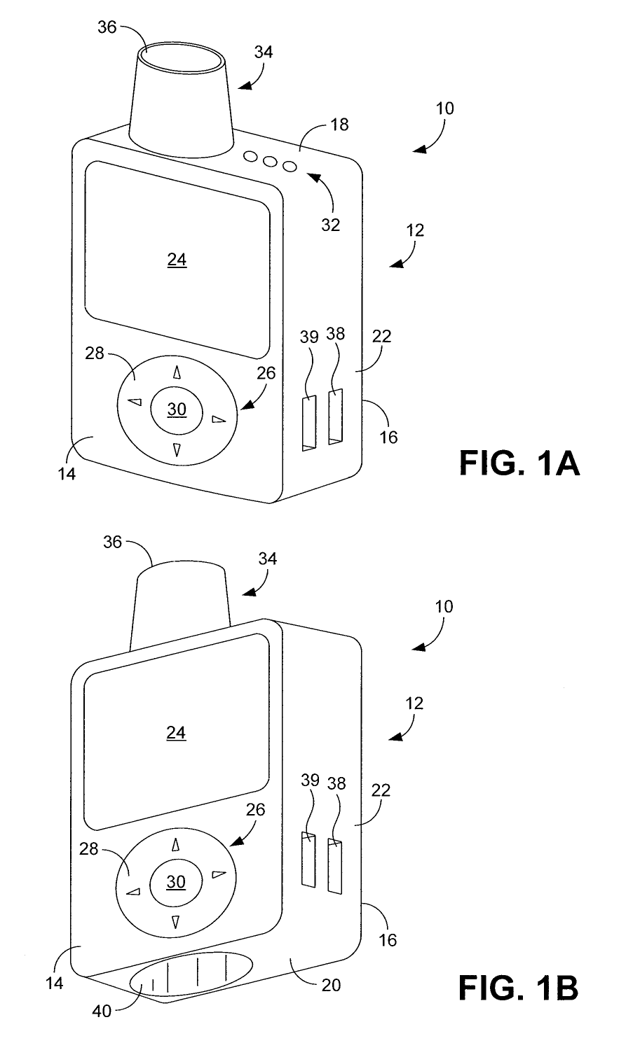

Referring to the drawings, in which like numerals indicate corresponding parts throughout the several views, FIGS. 1A and 1B illustrate a device 10 for diagnosing and treating a respiratory condition, such as an asthma attack. Although asthma has been specifically identified, it is noted that the device 10 can be used to diagnose and treat other respiratory conditions, including chronic obstructive airway disease (COAD). The device 10 may therefore be generally referred to as a respiratory condition device or a diagnosis/treatment device. In the embodiment of FIGS. 1A and 1B, the device 10 comprises a portable (e.g., handheld) device that can be easily carried with the user throughout the day so as to be available whenever needed. As indicated in the figures, the device 10 includes an outer housing 12 that generally comprises a front side 14, a rear side 16, a top side 18, a bottom side 20, and opposed lateral sides 22. Provided on the front side 14 is a user interface that, in the illustrated embodiment, includes display 24 and one or more input buttons 26. By way of example, the display 24 comprises a liquid crystal display (LCD) that can be used to communicate various information to the device user, such as instructions for use, results of any performed tests, or any other pertinent information. In the illustrated embodiment, the buttons 26 comprise a multi-directional button 28 and a select button 30 that can be used together to navigate screens and/or menus presented in the display 24 and make various selections. In some embodiments, the display 24 can be touch-sensitive such that the user selections can be directly entered with the display.

Provided on the top side 18 of the housing 12 in the illustrated embodiment are indicator lights 32 that can also convey various information to a user of the device 10. Such information may include various device conditions such as power on, low battery, or a ready state (e.g., ready to test and/or ready to administer medication). In some embodiments, the indicator lights 32 comprise light emitting diodes (LEDs). Also provided on the top side 18 of the housing 12 is a mouthpiece 34 that can be used to measure respiratory system parameters and to deliver medication to the respiratory system. In the embodiment of FIGS. 1A and 1B, the mouthpiece 34 comprises a hollow, frustoconical member that a user can place in his or her mouth. The mouthpiece 34 terminates in an opening 36 that, depending upon device operation, serves as an inlet or outlet for the device 10. More particularly, the opening 36 functions as an inlet during testing of the user's respiratory system and as an outlet during medication administration.

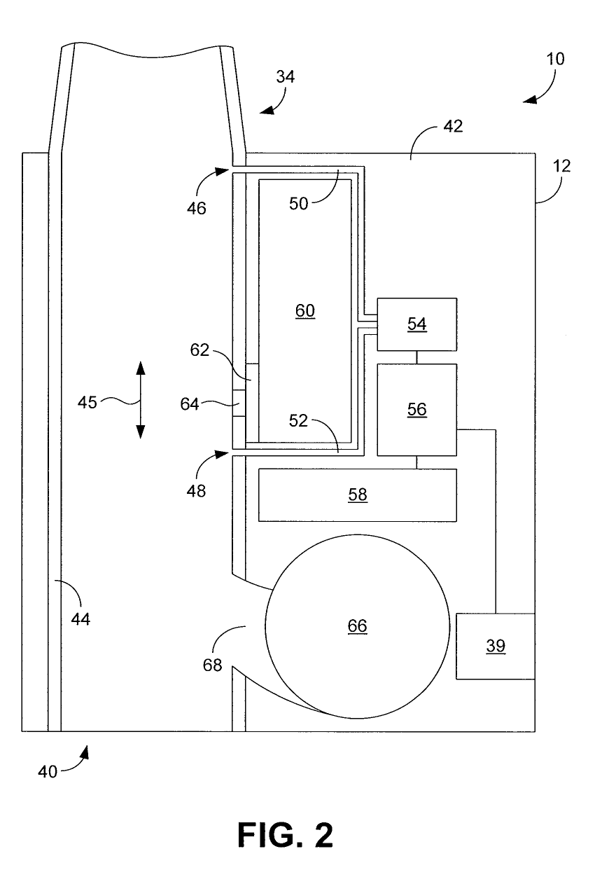

Provided on one of the lateral sides 22 of the housing 12 are electronic connectors 38 and 39. The connector 38 may comprise a power cable receptacle, such as an alternating current (AC) power cable receptacle, while the connector 39 may comprise a data cable connector, such as a universal serial bus (USB) cable connector, an IEEE 1394 (“FireWire”) cable connector, a parallel port, a serial port, or other connector. The connector 38 can be used to recharge an internal battery (FIG. 2) of the device 10. In some embodiments, the connector 39 can be used to communicate data obtained through respiratory system testing from the device 10 to a separate device, such as a physician's computer. In other embodiments, the connector 39 can be used to communicate to the device 10 data or logic for controlling operation of the device.

As indicated in FIG. 1B, provided on the bottom side 20 of the housing 12 is a further opening 40 that, depending upon device operation, serves as an outlet or inlet for the device 10. More particularly, the opening 40 functions as an outlet during testing of the user's respiratory system and as an inlet during medication administration.

FIG. 2 schematically illustrates an example architecture of the device 10 and, more particularly, components provided within an interior space 42 defined by the device outer housing 12. As indicated in FIG. 2, provided within the interior space 42 is a hollow drug delivery tube 44 that defines a flow path 45 of the device 10. The tube 44 extends from the mouthpiece 34 provided on the top side 18 to the opening 40 provided on the bottom side 20 of the housing 12. In the embodiment of FIG. 2, the tube 44 is generally straight and cylindrical. It is noted, however, that alternative shapes may be used. For example, the tube 44 can comprise a bend or curve. An example of a bent tube is described and illustrated in a copending patent application entitled “Apparatuses and Methods for Pulmonary Drug Delivery” and having Ser. No. 11/950,154, which is hereby incorporated by reference into the present disclosure in its entirety.

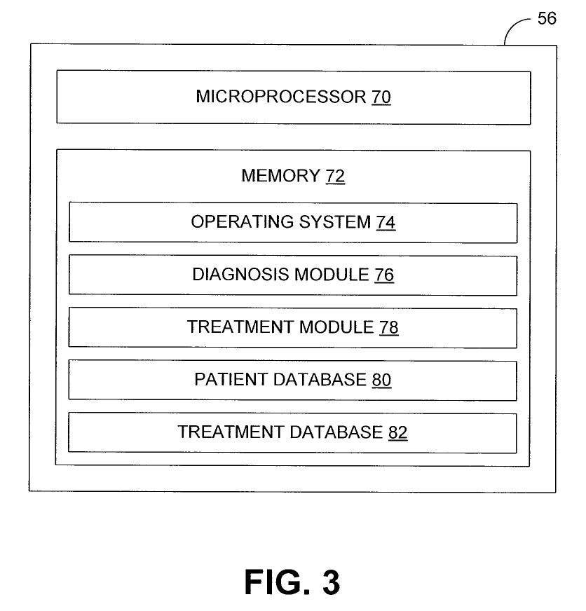

Formed within the drug delivery tube 44 are a first or upstream port 46 and a second or downstream port 48, both of which are in fluid communication with the tube and, therefore, the flow path 45. Each port 46, 48 is further in fluid communication with a signal tube 50, 52, each of which extends from its respective port to a differential pressure sensor 54. Through provision of the signal tubes 50, 52, fluid pressures at the first and second ports 46, 48, and therefore at different locations along the length of the tube 44, can be measured by the sensor 54. In some embodiments, the sensor 54 comprises a silicon-based differential pressure sensor. In some embodiments, the sensor 54 can measure flow characteristics of the user's respiratory system with the same or greater accuracy of a physician's spirometer. Signals indicative of the observed pressures can be provided from the sensor 54 to a controller 56, which controls the overall operation of the device 10. An example embodiment of the controller 56 is provided in relation to FIG. 3 below.

Also provided within the interior space 42 is an internal power source 58, such as battery. In embodiments in which the device 10 includes a power cable receptacle, the power source 58 can comprise a rechargeable battery. In other embodiments, the power source 58 can comprise a conventional disposable battery that is replaced when exhausted.

The device 10 illustrated in FIG. 2 further comprises a medicine container 60 that is used to hold medicine that can be administered to the device user. In some embodiments, the medicine comprises a liquid medicine solution that includes a drug that is used to treat a respiratory condition, such as an asthma attack. Example drugs that may be administered to a patient include bronchodilators (e.g., albuterol) and corticosteroids. In alternative embodiments, the medicine can comprise powered medicine.

Irrespective of the nature of the medicine, the container 60 supplies the medicine to a medicine delivery device 62, which delivers medicine into the drug delivery tube 44, and therefore flow path 45, via a supply port 64. In some embodiments, the medicine delivery device 62 comprises a droplet ejection device similar to those used in inkjet printers. In such an embodiment, the medication delivery device 62 is used to selectively eject fine droplets of medication into the tube 44 from firing chambers of the droplet ejection device. When it is desired to eject medicine from the droplet ejection device, droplet ejection elements provided in or adjacent the chambers are energized. In embodiments in which the droplet ejection elements comprise heater resistors, thin layers of the medicine within the firing chambers are superheated, causing explosive vaporization and ejection of droplets of medicine through nozzles of the droplet ejection device and then through the supply port 64. Ejection of the droplets creates a capillary action that draws further medicine within the firing chambers of the droplet ejection device such that the droplet ejection device can be repeatedly fired.

In alternative embodiments, the medicine delivery device 62 comprises a device that atomizes liquid medicine, such as a nebulizer. In further alternative embodiments, the medicine delivery device 62 comprises a device that fluidizes powdered medicine, such as a mechanical agitator.

As is further indicated in FIG. 2, the device 10 includes an internal fan 66 that can be used to increase the velocity of air and medication during treatment of a respiratory condition. In some embodiments, the fan 66 comprises a centrifugal fan that is selectively operated in response to detection of user inhalation during a treatment phase of device operation. Air can be drawn in by the fan 66 through an inlet (not shown) provided on the device 10 (e.g., on its rear side) and can be input into the flow path 45 of the drug delivery tube 44 via an outlet 68.

In the above description, the device 10 is described as comprising a single medicine container and medicine delivery device. It is noted, however, that the device 10 can alternatively comprise multiple medicine containers and medicine delivery devices. In such cases, multiple different medicines can be selectively administered to the user, if desired.

FIG. 3 illustrates an example embodiment for the controller 56 shown in FIG. 2. The controller 56 of FIG. 3 comprises a microprocessor 70 and memory 72, which can in some embodiments form part of the microprocessor. Stored within memory 72 is various logic, including an operating system 74 that is used to control overall operation of the device 10 and the other programs and/or modules stored within memory. In addition, the memory 72 includes a diagnosis module 76 that is used to diagnose one or more respiratory conditions. In some embodiments, the diagnosis module 76 is configured to determine whether the user's respiratory tract is in a state that indicates that the user is having or about to have an asthma attack. In further embodiments, the diagnosis module 76 can determine the severity of the asthma attack.

Also stored within memory 72 is a treatment module 78 that is used to control treatment of the user with the device 10. More particularly, the treatment module 78 controls the administration of medication to the user, for example when data obtained through a testing phase of device operation indicates that a given respiratory condition is occurring or is about to occur. In some embodiments, the treatment module 78 uses volumetric measurements obtained by the diagnosis module 76 to identify, through reference to a treatment database 82, whether administration of medicine is warranted and, if so, at what dosage. The treatment module 78 can then control the medicine delivery device 62 and the fan 66 to deliver the indicated medicine dosage to the user. Furthermore, the memory 72 can include a patient database 80 in which measured parameters of the user can be stored.

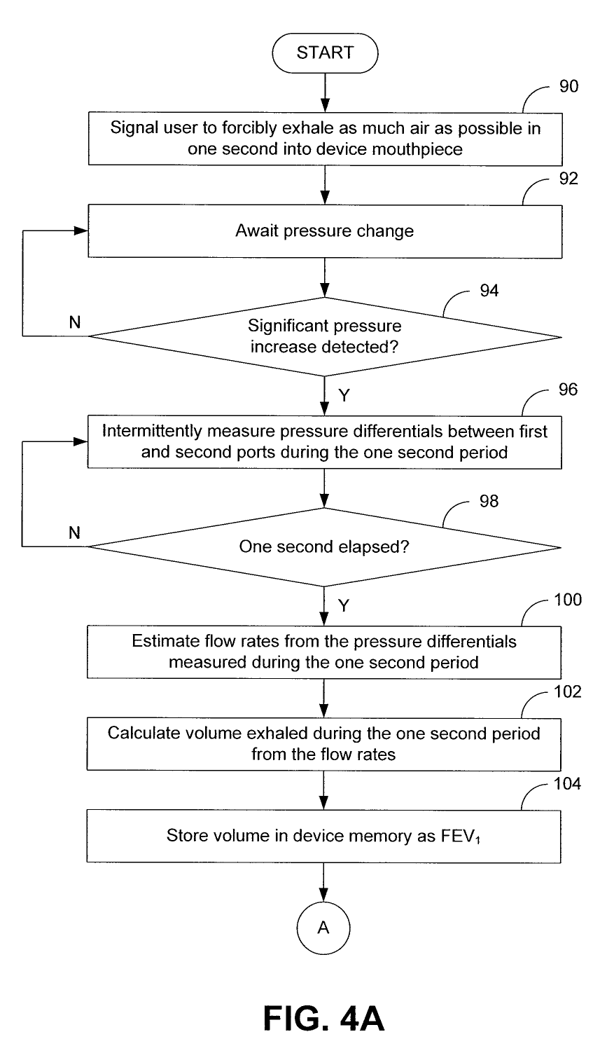

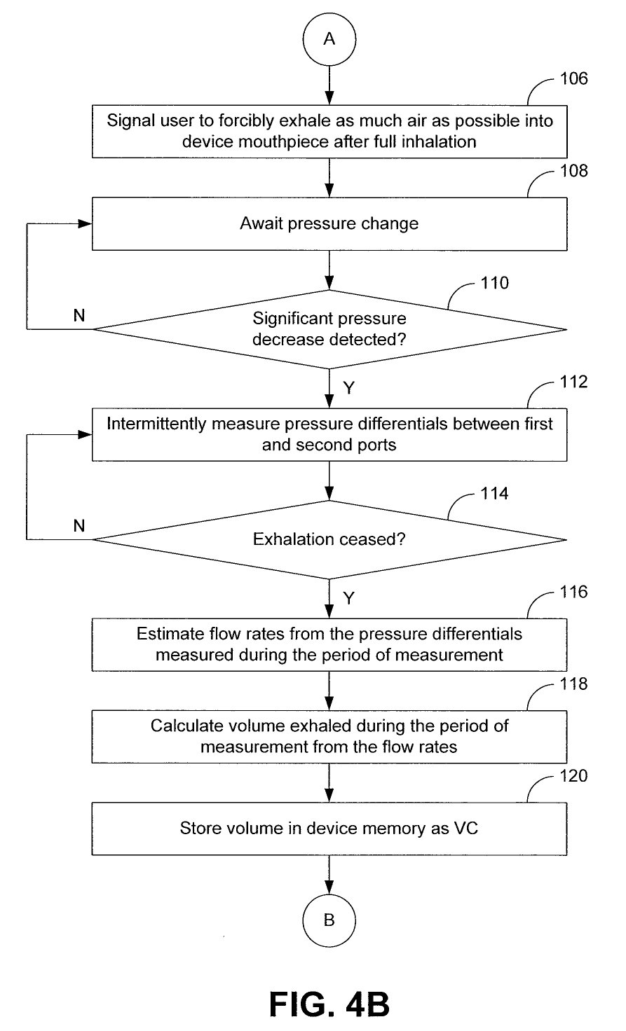

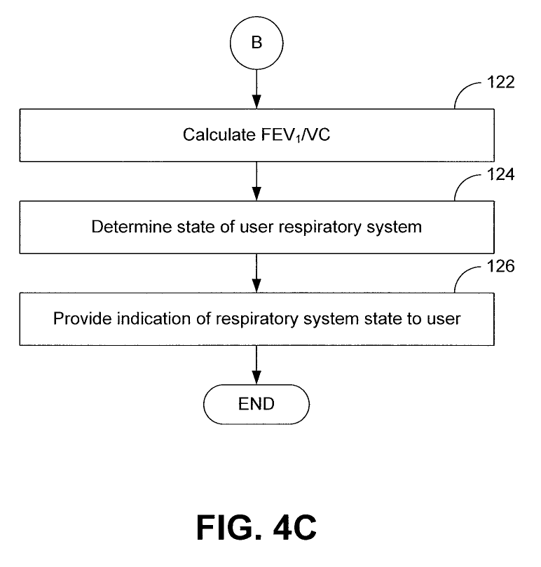

FIGS. 4A-4C illustrate an example method for diagnosing a respiratory condition using a device, such as the device 10 described in the foregoing. In some embodiments, the various actions described in the example method are performed by or under the direction of the diagnosis module 76. As described in the following, the device 10 is used to measure volumes of exhalation of the user and, from those volumes, determine the state of the user's respiratory system. For example, the device 10 can determine the ratio between the user's forced expiratory volume in one second (FEV₁) and the user's vital capacity (VC), denoted herein as FEV₁/VC. That ratio can then be used to diagnose a given respiratory condition, such as an asthma attack.

When the user wishes to be tested to determine whether he or she is experiencing an asthma attack, the user initiates a test mode on the device. By way of example, the test mode of operation can be initiated by selecting an appropriate command displayed to the user in the device display using the input buttons described above. Once the test mode has been initiated, the device signals the user to forcibly exhale as much air as possible in one second into the device mouthpiece, as indicated in block 90 of FIG. 4A. In some embodiments, the device can signal the user to so exhale using the device display. For example, the device display can provide textual and/or graphical instructions to the user that explain how the user is to exhale into the device and the need to exhale as much volume of air as the user can within a one second time period. In other embodiments, the device can signal the user with one or more of the indicator lights provided on the device housing.

After signaling the user to begin exhaling, the device awaits a pressure change (block 92) indicative of the user blowing into the mouthpiece. The pressure change can be detected using the differential pressure sensor provided within the device. With reference to decision block 94, if no significant pressure increase is detected, the process returns to block 92 at which the device continues to await a pressure change. Notably, the device can time out of the test mode if a significant pressure increase is not detected for an extended period of time. If a significant pressure increase is detected, however, meaning a pressure change is observed that is greater than that which may be observed due to normal fluctuation in ambient conditions, the device immediately begins intermittently measuring pressure differentials between the first and second ports of the device during the one second period, as indicated in block 96. Notably, the pressure differentials are at least temporarily stored in device memory for use in calculating flow rates and exhaled volumes, as described below.

Referring to decision block 98, if a full second has not elapsed, the process returns to block 96 and further pressure differentials are measured. If one second has elapsed, however, the process continues to block 100 at which the device estimates flow rates from the measured pressure differentials. In one embodiment, the flow rates are estimated using Ohm's law (as adapted for fluid flow) and Poiseuille's law. Ohm's law may be stated as follows:

ΔP=V·R [Equation 1]

wherein V· is flow rate, ΔP is the pressure differential, and R is a resistance value indicative of the resistance provided by the walls of the tube that define the flow path. R can be determined for a cylindrical pipe or tube from Poiseuille's law, which may be stated as follows:

$$R = \frac{8 nl}{π {r}^{4}} \qquad [ Equation 2 ]$$

where n is the viscosity of the fluid, l is the length of separation between the points at which the pressures were measured, and r is the radius of the pipe or tube. Therefore, the flow rate can be estimated as:

$${V}^{•} = \frac{Δ P π {r}^{4}}{8 nl} \qquad [ Equation 3 ]$$

Once the flow rates have been estimated for each pressure differential, the volume exhaled during the one second period can be calculated, as indicated in block 102. In some embodiments, such calculation comprises performing an integration using the various observed flow rates. Expressed in a mathematical sense, the volume is equal to the area under a curve defined by the various flow rate data points. After the volume has been calculated, the volume is then stored in device memory as FEV₁, as indicated in block 104.

At this point, one of the two exhalation volumes to be used to diagnose has been determined. Next, VC is determined. Referring to block 106 of FIG. 4B, the device signals the user to forcibly exhale as much air as possible into the device mouthpiece after full inhalation. Again, the device can signal the user using the device display and/or the indicator lights. After signaling the user to begin exhaling, the device awaits a pressure change (block 108) indicative of the user blowing into the mouthpiece. With reference to decision block 110, if no significant pressure increase is detected, the process returns to block 108 at which the device continues to await a pressure change. If, on the other hand, a significant pressure increase is detected, the device immediately begins intermittently measuring pressure differentials between the first and second ports of the device, as indicated in block 112. As before, the pressure differentials are at least temporarily stored in device memory.

Referring to decision block 114, it is determined whether exhalation has ceased. In some embodiments, the exhalation cessation determination is made by detecting reduction of pressures to levels observed prior to user exhalation (i.e., return to ambient conditions). In other embodiments, the user can affirmatively signal that he or she is done exhaling using the device user interface. If exhalation has not ceased, the process returns to block 112 at which measurement of pressure differentials continues. If, however, exhalation has ceased, the process continues to block 116 at which the device estimates flow rates from the measured pressure differentials. In some embodiments, the estimate is performed using Ohm's law and Poiseuille's law in the manner described above.

Once the flow rates have been estimated, the total volume of air exhaled by the user during the period of measurement can be calculated, as indicated in block 118. In some embodiments, such calculation comprises performing an integration in the manner described above. After this new volume has been calculated, the volume is then stored in device memory as VC, as indicated in block 120. Although separate exhalations have been described as being used to obtain FEV₁ and VC, it is noted that, in alternative embodiments, a single exhalation cycle could instead be used to determine both FEV₁ and VC.

With reference next to block 122 of FIG. 4C, the device then calculates FEV₁/VC through simple division. At this point, the device has determined the ratio between the user's forced expiratory volume in one second and the user's vital capacity, which is a good indicator of respiratory system health and whether the user is having an asthma attack. When the user is not having an asthma attack, FEV₁/VC will typically be in the range of approximately 75% to 80%. When FEV₁/VC falls below 75%, however, particularly when FEV₁/VC falls to value significantly lower that 75%, a respiratory system state in which an asthma attack is occurring or is about to occur may be assumed.

Referring next to block 124, the device can determine the state of the user respiratory system. In some embodiments, the device can be calibrated, for example by a physician, to set the point at which a positive indication for a given respiratory condition will be determined. For instance, the device may be calibrated to indicate an asthma attack if FEV₁/VC is determined to be below 70%, 60%, 50%, or some other ratio. Next, the device can provide the user with an indication of the respiratory system state, as indicated in block 126. In some embodiments, the device can simply indicate that conditions are such that an asthma attack is or is not occurring (i.e., positive or negative). In other embodiments, the device can provide the user with a quantitative and/or qualitative indication of the current health of the user's respiratory system. For example, the device can display the FEV₁/VC ratio to the user and indicate whether that ratio translates to good, fair, or poor respiratory system health. Such information can be conveyed to the user with text and/or graphics in the device display.

In the above description, a pressure sensor is used to determine FEV₁/VC. It is noted that, in alternative embodiments, FEV₁/VC can be determined, at least in part, using the fan of the diagnosis/treatment device. Specifically, in embodiments in which the fan comprises an integrated tachometer and the fan can freely spin in response to the user's exhalation into the drug delivery tube, the rate of fan revolution can be measured to determine the speed or velocity of the air flow over time. From that information and flow calibration data, the volume of exhalation, and therefore, FEV₁ and VC, can be calculated. In such a case, the pressure sensor may not be needed and therefore may excluded from the device. In further alternative embodiments, the diagnosis/treatment device can be provided with a flow meter in the form of a hot wire anemometer. When a hot wire anemometer is used, its wire can be provided within the flow path of the drug delivery tube such that the speed or velocity of the user's exhalation can likewise be measured over time to determine the volume of air that the user has exhaled. In such a case, the pressure sensor also may not be necessary.

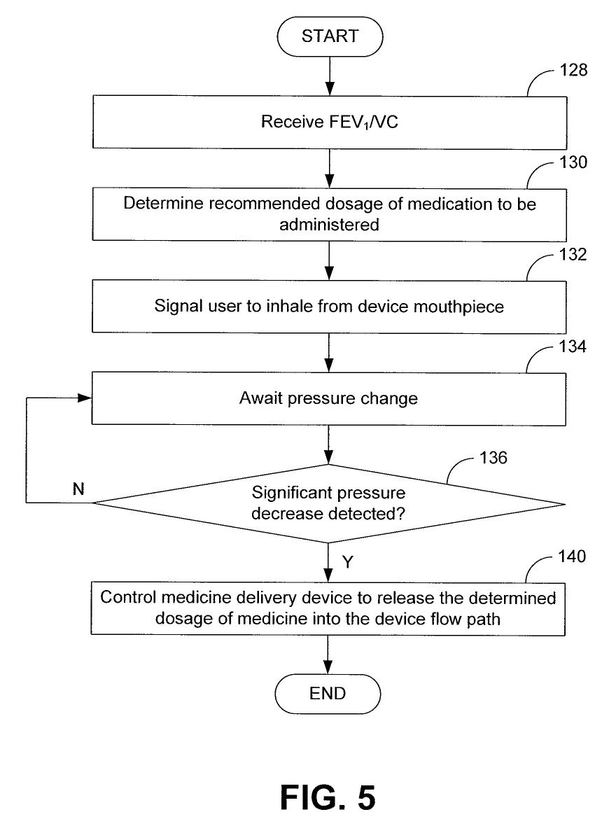

FIG. 5 illustrates an example method for treating a respiratory condition using a device, such as the device 10 described in the foregoing. In some embodiments, the various actions described in the example method are performed by or under the direction of the treatment module 78 after the process described above in relation to FIGS. 4A-4C has been performed. In the example method of FIG. 5, the device 10 is used to deliver a desired amount of medicine to the flow path defined by the device for inhalation by the user (patient). The medicine can be added to the flow path as liquid droplets or as dry powder particles.

Beginning with block 128 of FIG. 5, the FEV₁/VC ratio obtained through the testing mode of operation of the device is received. That ratio can be used to determine whether administration of medicine is warranted and, in some embodiments, the dosage of medicine that is to be administered. Therefore, as indicated in block 130, the device determines the dosage of medication that is recommended from FEV₁/VC. In some embodiments, the dosage is determined through reference to a lookup table that cross-references FEV₁/VC ratios with medication dosages. Notably, the recommended dosage can be no dosage at all. In further embodiments, the recommended dosage may be dependent upon other factors beyond FEV₁/VC. In some cases, the age and/or sex of the user can be considered when determining dosage. In other cases, other drugs that the user is already taking are considered. Such other factors can also be accounted for in the lookup table. To cite an example, the lookup table can identify FEV₁/VC ratio along the x axis and age along the y axis and the intersection of the relevant values can identify the recommended dosage. Regardless, the factors and associated dosages can be preprogrammed into the device, for example by a physician, and stored in the device's treatment database. It is noted that, in other embodiments, one or more appropriate algorithms can be used in lieu of a lookup table to custom tailor the medication dosage for the user.

Once the recommended dosage has been determined, the device signals the user to inhale from the device mouthpiece, as indicated in block 132. In some embodiments, the device can signal the user to so inhale using the device display. For example, the device display can provide textual and/or graphical instructions to the user that explains how the user is to inhale from the device and for what duration. In addition, the device display can provide an indication as to how long the inhaled breath should be held to obtain optimal absorption of the medication. In other embodiments, the device can signal the user with one or more of the indicator lights provided on the device housing.

Next, the device awaits a pressure change, as indicated in block 134. Referring to decision block 136, if no significant pressure decrease is detected, flow returns to block 134 and the device continues to wait for a pressure change. If a significant pressure decrease is detected, however, the device controls the medicine delivery device to release the determined dosage of medicine into the device flow path such that the user will inhale the medicine. If the device comprises a fan, the fan is operated substantially simultaneously to assist in delivery of the medicine. In embodiments in which the medicine is stored in liquid form within the device, droplets of medicine are ejected into the flow path. In embodiments in which the medicine is stored in powder form within the device, dry particles of medicine are ejected into the flow path. Notably, more than one medication can be administered in cases in which the device includes multiple medicine containers and medicine delivery devices.

From the above it can be appreciated that the diagnosis/treatment device can be used to provide a patient, or the patient's guardian (e.g., parent) with a clear indication as to whether medicine should be administered to treat a respiratory condition, such as an asthma attack, thereby removing the guesswork out of the determination. Furthermore, that indication is provided with a convenient, portable device that can be carried with the patient at substantially all times so that a determination can be made whenever the patient or guardian believes there may be a need to evaluate the patient's respiratory system. Moreover, when treatment is needed, the device provides for such treatment and, in some embodiments, automatically determines the correct dosage to be administered relative to the results of patient testing. Accordingly, more appropriate dosages can be administered relative to the current condition of the patient, in contrast to the “one dosage fits all” administration scheme currently used by most asthmatics.

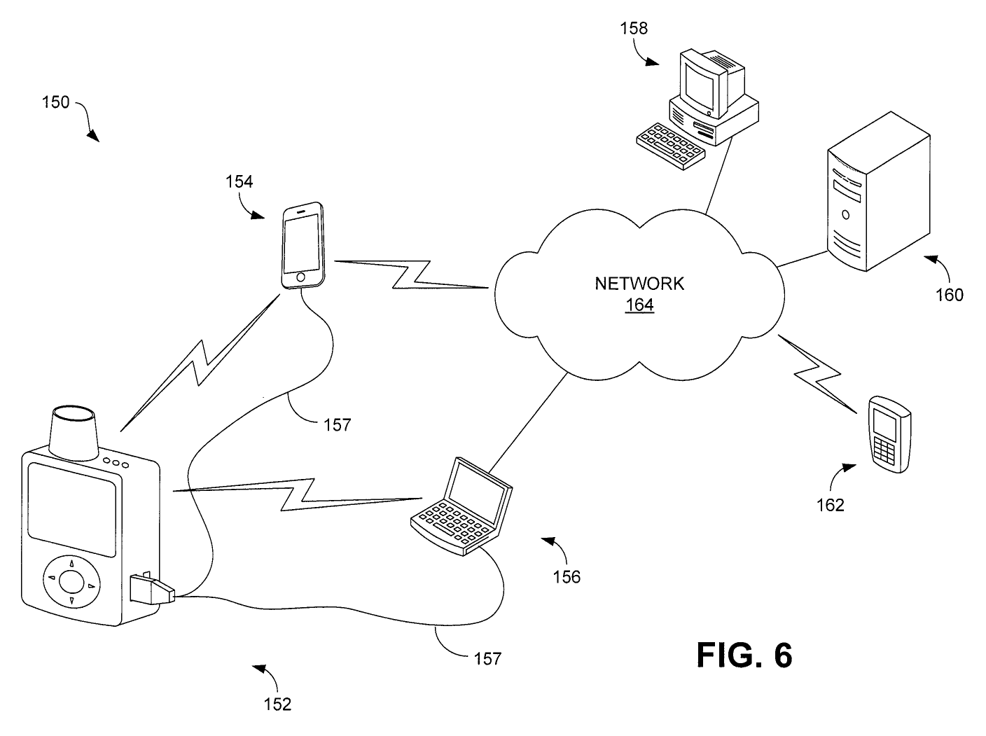

Devices of the type described above can be used to serve other functions beyond diagnosing and treating a respiratory condition. In particular, when the FEV₁ and VC values are stored in device memory as described above in relation to FIGS. 4A-4C, the health of the patient's respiratory system over an extended time period can be mapped for later consideration. For instance, if the patient were to test his or her respiratory system health on a continual periodic basis, such as daily, the data collected from each day could be stored and periodically transmitted to the patient's physician for review. Such information would be valuable to the physician in evaluating the patient's respiratory function and diagnosing the severity of the patient's condition (e.g., asthma). Such information therefore could assist the physician in determining the most appropriate treatment for the patient. In another example, appropriate persons, such as relatives, physicians, emergency services (e.g., “911”), or emergency medical technicians (EMTs) can be alerted when emergency conditions are detected through the diagnosis process. Such additional functionalities are facilitated with the system described in relation to FIG. 6.

In an effort to increase the accuracy with which respiratory conditions are diagnosed by the diagnosis/treatment device, the device can be calibrated prior to it being provided to a patient. By way of example, such calibration can be performed by the patient's physician. In one embodiment, the physician can calibrate the diagnosis/treatment device by comparing values measured by the device with values measured by a high-precision device used by the physician during office visits. For instance, the patient's VC can be measured with both the diagnosis/treatment device and a spirometer and the results compared. If it is determined that the measurement from the diagnosis/treatment device significantly differs from that of the spirometer, which is generally considered to be a high-precision device, adjustments can be made to the diagnosis/treatment device to compensate for its error. Such adjustments may include the adjustment of one or more variables of an algorithm used to determine the respiratory parameter. For example, the value for R in Ohm's law discussed above can be adjusted.

In another embodiment, the diagnosis/treatment device can be calibrated by measuring a known parameter, such as a known volume of expelled air. For instance, the diagnosis/treatment device can be connected to a mass flow meter that can be programmed to expel a given quantity of air. In cases in which the mass flow meter is a high-precision device, deviation of the diagnosis/treatment device's measurements and the volume actually expelled by the mass flow meter can be presumed to be device inaccuracy and, as described above, adjustments can be made to the diagnosis/treatment device to compensate for its error.

It is further noted that, in some embodiments, a high-precision measurement device, such as a spirometer, can be used to measure patient parameters that are not expected to fluctuate and such parameters can be stored in the diagnosis/treatment device for later use. For example, a spirometer can be used to measure the patient's VC, which should remain substantially the same irrespective of whether the patient is or is not experiencing an asthma attack. The VC value can then be uploaded to the diagnosis/treatment device for storage and usage in diagnosis and treatment. In such cases, the diagnosis/treatment device need only measure FEC₁, which only requires a one-second exhalation from the patient.

FIG. 6 illustrates an example of a system 150 for diagnosing and treating a respiratory condition, such as an asthma attack. The system 150 uses a diagnosis/treatment device 152 that is similar to the device 10 described in the foregoing. The device 152, however, can leverage the functionality of another device to provide one or more of user interfacing, diagnosis/treatment control, and communication of information to remote persons. By way of example, those functions are provided by a separate device owned, possessed, or simply accessed by the user, such as a mobile telephone 154 or a computer 156 (e.g., laptop or personal computer (PC)). Although a mobile “telephone” has been identified, it is to be understood that such a telephone may be an integrated device that comprises one or more functionalities of another device, such as a computer or a music player. For example, the mobile telephone 154 can comprise a personal digital assistant, such as a Treo™ or a BlackBerry™, or can comprise a multimedia device, such as an iPhone™. Furthermore, although a telephone and a computer have been explicitly identified, it is to be appreciated that substantially any other device can be used, assuming it is capable of communicating with the device 152 and can provide an added functionality for the user. Irrespective of the nature of the separate device, the separate device can, in some embodiments, comprise the logic that performs the functions associated with the diagnosis module 76 and/or treatment module 78 described above. In such embodiments, the memory of the device 152 can be limited to storing an operating system that controls data collection/transmission and delivery of medicine.

As is further indicated in FIG. 6, the device 152 can communicate with the separate devices 154, 156 with a data cable 157 and/or wirelessly. The data cable 157 can comprise, for example, a USB or FireWire cable. In some embodiments, wireless communications comprise short-range radio frequency (RF) communications, for example using one or more of the Bluetooth, IEEE 802.15.4 (“ZigBee”), or IEEE 802.11 (“Wi-Fi”) protocols. With the ability to communicate via a cable or wirelessly, the device 152 can collect various data during patient testing, such as exhalation volumes, and provide the data to one of the separate devices 154, 156. The separate device 154, 156 can then determine the condition of the patient's respiratory system and the correct dosage of medicine to administer (if any), and send instructions to the device 152 that indicate the dosage of medication (if any) that should be administered.

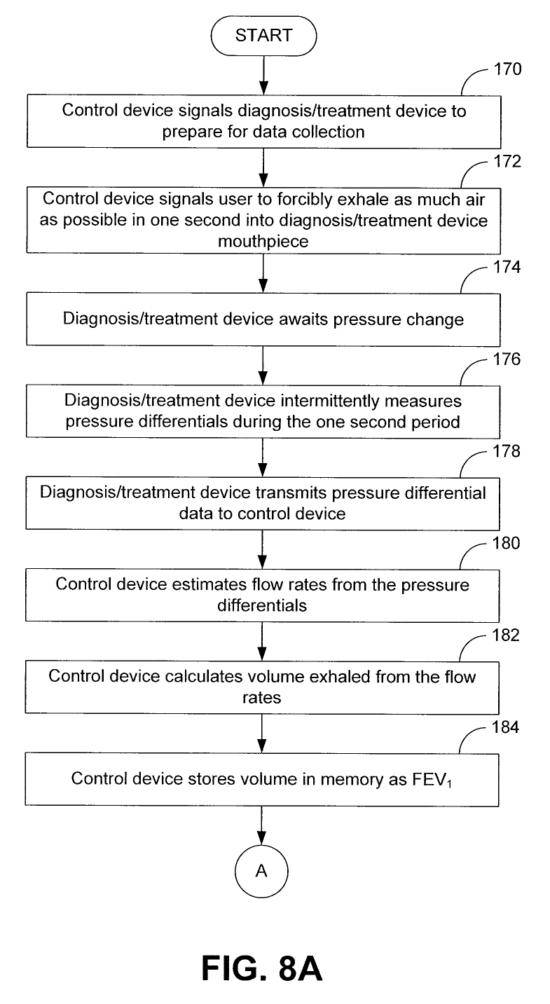

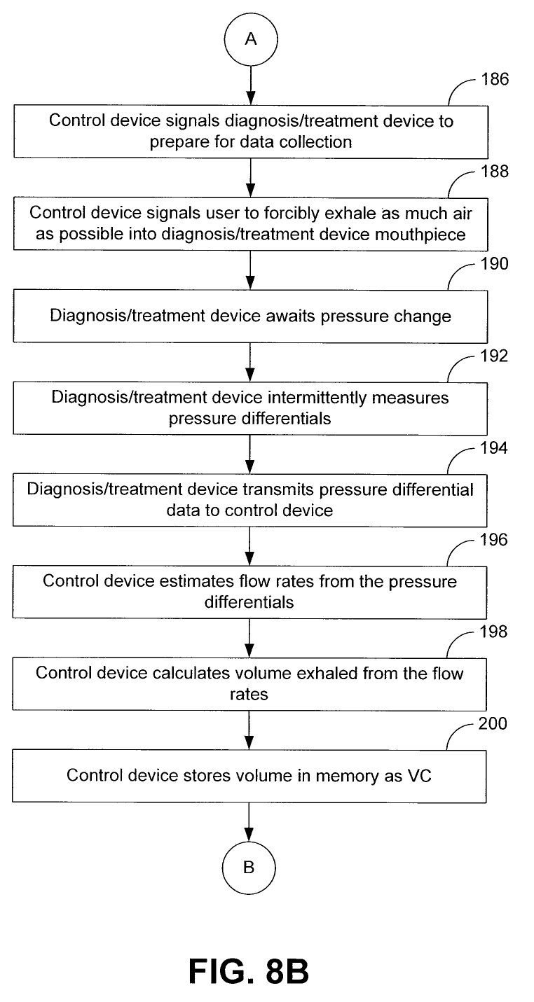

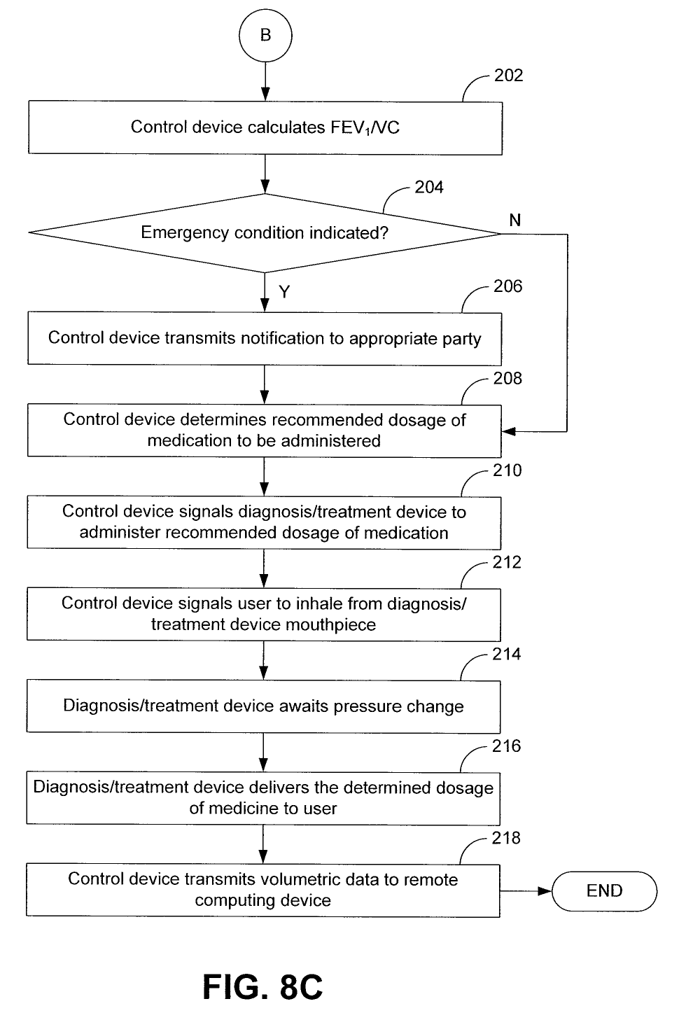

In addition or exception to controlling the device 152, the separate device 154, 156 can share data with one or more remote devices, such as a desktop computer 158, a data storage computer 160, or a mobile telephone 162. In some embodiments, one or more of the remote devices can be operated by the patient's physician or guardian. In other embodiments, one or more of the remote devices can be operated by an emergency service. Therefore, data obtained by and/or diagnoses determined by the device 152 and/or the separate device 154, 156 can be shared with persons who are remote from the patient, for example via a network 164. The network 164 may comprise a telephone service network and/or a computer network that forms part of the Internet. An example method of use of the system 150 is provided in relation to FIGS. 8A-8C below.

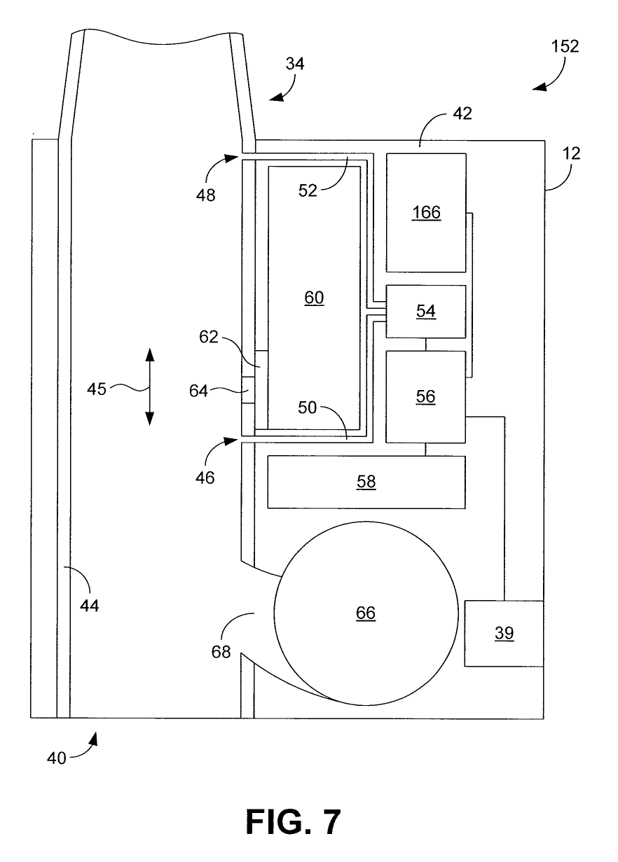

FIG. 7 schematically illustrates an example construction of the diagnosis/treatment device 152 and, more particularly, components provided within an interior space 42 defined by the device outer housing 12. As is apparent from FIG. 7, the device 152 comprises several of the same components of the device 10. Therefore, the device 152 includes a drug delivery tube 44 that defines a flow path 45 and first and second ports 46, 48 that are in fluid communication with the flow path and signal tubes 50, 52 that extend to a differential pressure sensor 54. Furthermore, the device 152 includes a controller 56 that controls the overall operation of the device 152, a data cable connector 39, a power source 58 that provides power to the device, a medicine container 60 that is used to hold medicine that can be administered to the device user, and a medicine delivery device 62 that delivers medicine to the flow path 45 via a supply port 64. Moreover, the device 152 includes a fan 66 that can be used to blow air into the flow path 45 via an outlet 68 to increase the velocity of air and medication during treatment of a respiratory condition.

In addition to the above-described components, the device 152 further includes a wireless transceiver 166 that can be used to wirelessly transmit data to a separate device and wirelessly receive instructions from the separate device. In some embodiments, the transceiver 166 comprises an RF transceiver, such as a Bluetooth, ZigBee, or Wi-Fi transceiver.

FIGS. 8A-8C illustrate an example of operation of the system 150 of FIG. 6. In the described method, a respiratory system condition, such as an asthma attack, is diagnosed and treated using the diagnosis/treatment device 152. In addition, parameters measured during diagnosis are collected on a separate device and periodically provided to a remote computing device, such as a computer operated by the patient's physician. Furthermore, detected emergency medical conditions are communicated to one or more appropriate parties, such as the patient's guardian and/or an emergency medical service.

When the user wishes to be tested to determine whether he or she is experiencing an asthma attack, the user initiates a testing or diagnosis process. In this example, it is assumed that the process is controlled by the separate device (e.g., mobile phone 154 or computer 156). Therefore, for the remainder of the discussion of FIGS. 8A-8C, the separate device will be referred to as the “control device” in recognition of its control function relative to the diagnosis/treatment device. It is noted, however, that control can instead be retained by the diagnosis/treatment device 152, in which case the separate device would be primarily used to communicate information from the diagnosis/treatment device to a remote device (e.g., via the network 164).

Once a test process has been initiated, the control device signals the diagnosis/treatment device to prepare for data collection, as indicated in block 170 of FIG. 8A. In addition, the control device signals the user to forcibly exhale as much air as possible in one second into the diagnosis/treatment device mouthpiece, as indicated in block 172. In some embodiments, the control device can signal the user to so exhale using a display of the control device. For example, the control device display can provide textual and/or graphical instructions to the user that explains how the user is to exhale into the device and the need to exhale as much volume of air as the user can within a one second time period.

In response to the signal from the control device, the diagnosis/treatment device awaits a pressure change, as indicated in block 174. Once a significant pressure increase is detected, the diagnosis/treatment device immediately begins intermittently measuring pressure differentials between the first and second ports of the device during the one second period, as indicated in block 176. The pressure differentials are at least temporarily stored in diagnosis/treatment device memory.

Assuming that the control device will make the determination as to the health of the user's respiratory system, the pressure differential data is transmitted from the diagnosis/treatment device to the control device as or after the pressure differentials are measured, as indicated in block 178. The control device then estimates flow rates from the measured pressure differentials (block 180) and calculates exhaled volume from the flow rates (block 182) in similar manner to that described above in relation to FIGS. 4A-4C. After the volume has been calculated, the volume is then stored on the control device as FEV₁, as indicated in block 184.

Referring next to block 186 of FIG. 8B, the control device again signals the diagnosis/treatment device to prepare for data collection. In addition, the control device signals the user to forcibly exhale as much air as possible into the diagnosis/treatment device mouthpiece after full inhalation, as indicated in block 188. Again, the control device can signal the user using the control device display. The diagnosis/treatment device again awaits a pressure change (block 190) and, once a significant pressure increase is detected, the diagnosis/treatment device immediately begins intermittently measuring pressure differentials (block 192). The pressure differentials are at least temporarily stored in diagnosis/treatment device memory.

As or after the pressure differentials are measured, the pressure differential data is transmitted from the diagnosis/treatment device to the control device, as indicated in block 194. The control device then estimates flow rates from the measured pressure differentials (block 196) and calculates exhaled volume from the flow rates (block 198) in similar manner to that described above in relation to FIGS. 4A-4C. After the volume has been calculated, the volume is then stored on the control device as VC, as indicated in block 200.

Referring next to block 202 of FIG. 8C, the control device calculates FEV₁/VC. At this point, the device has determined the ratio between the user's forced expiratory volume in one second and the user's vital capacity, which is a good indicator of whether the user is having an asthma attack. Given that the FEV₁/VC value provides an indication of the health of the user's respiratory system, the control device can determine whether an emergency condition is indicated. By way of example, an emergency condition comprises a condition in which FEV₁/VC is so low as to indicate that the bronchial passages are constricted to a point at which there is a risk of asphyxiation. In such a case, it may be desirable to communicate the user's condition to others so that appropriate action can be taken. With reference then to decision block 204, if an emergency condition is indicated, the process continues to block 206 at which the control device transmits a notification (e.g., alert) to an appropriate party. The party that receives the notification can be programmed by the user or the user's guardian. By way of example, appropriate parties may comprise one or more of the user's guardian, the user's physician, emergency services (e.g., “911”), emergency medical service providers, hospitals, and the like. The nature of the notification can also be preprogrammed by the user or the user's guardian. In some embodiments, the notification comprises an electronic alert sent to a remote computer. In other embodiments, the notification comprises an email message sent to a remote computer. In further embodiments, the notification comprises a text message sent to a remote mobile telephone. In still further embodiments, the notification comprises a phone call and recorded phone message sent to a remote mobile telephone.

After a notification has been sent, or if no such notification was necessary, the process continues to block 208 at which the control device determines a recommended dosage of medication to be administered. The dosage can, in some embodiments, be determined through reference to a lookup table that cross-references FEV₁/VC ratios with dosages. Again, the recommended dosage may be dependent upon other factors beyond FEV₁/VC. Once the recommended dosage has been determined, the control device signals the diagnosis/treatment device to administer the recommended dosage of medication, as indicated in block 210, and signals the user to inhale from the device mouthpiece, as indicated in block 212. The diagnosis/treatment device then awaits a pressure change, as indicated in block 214, and once a significant pressure decrease is detected, the diagnosis/treatment device controls the medicine delivery device to release the determined dosage of medicine into the device flow path such that the user will inhale the medicine, as indicated in block 216.

At this point, treatment has been provided to the user. Given that valuable data has been collected about the health of the user's respiratory system, it may be useful to provide that data to a physician so that the physician may better understand how well or how poorly the user's respiratory system is functioning. With such information, the physician can make a better informed decision as to treatment. To that end, the control device transmits the volumetric data obtained through the diagnosis process to a remote computing device, such as a physician's desktop computer or data storage computer, as indicated in block 218.

Again, it is noted that, although a separate device (e.g., mobile telephone 154 or computer 156) can provide control functionality as described in relation to FIGS. 8A-8C above, in other embodiments only the network communication functionalities of the separate device are leveraged. In such cases, the diagnosis/treatment device 152 controls testing, makes respiratory system health determinations, and provides data and/or messages to the separate device for transmission to a remote device. When data or a message is to be transmitted, the device 152 can also identify to the separate device the telephone number(s) and/or electronic address(es) of the intended recipient(s).

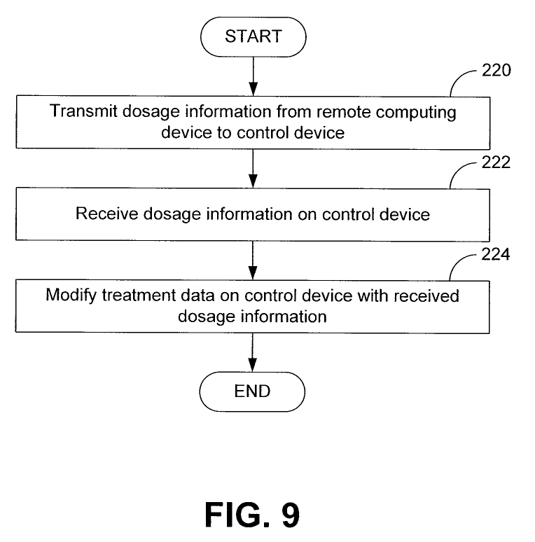

When the volumetric information is provided to a physician, there are various actions that the physician can take. One such action is adjusting a patient's dosage. For example, if the physician initially underestimated the severity of a patient's asthma, but later appreciates the level of severity in view of the volumetric information recorded over a period of time, the physician may determine to increase the dosage of medication that is administered relative to the observed FEV₁/VC ratios. Although such an adjustment could be made by physically connecting the diagnosis/treatment device or the applicable control device to the physician's computer and uploading new dosage information to the device, such a solution requires a visit to the physician's office. More desirable would be enabling the physician to control dosage remotely. FIG. 9 considers an example of one such control scheme.

Beginning with block 220 of FIG. 9, a physician transmits dosage information from a remote computing device to the control device. In some embodiments, the dosage information can comprise, for example, new recommended dosages relative to FEV₁/VC ratios that may be observed. In embodiments in which the control device is a computer, the dosage information can, for example, be sent as an executable or text file attached to an email message. In embodiments in which the control device is a mobile telephone, the dosage information can be sent as an executable or text file attached to an email message or a text message.

Once the dosage information is transmitted, it is received by the control device, as indicated in block 222. The control device can then modify treatment data, for example treatment data stored in a treatment database of the control device, with the received dosage information, as indicated in block 224. With such modification, new dosages of medication can be administered for observed values of FEV₁/VC.

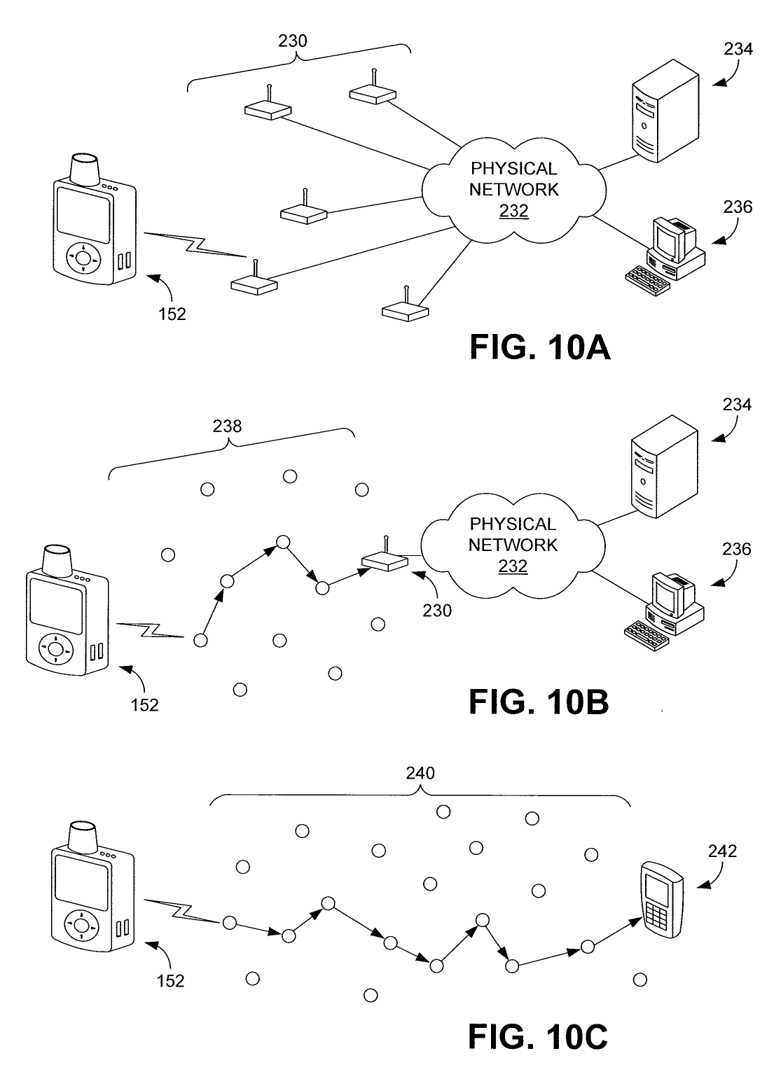

In the embodiments described in relation to FIGS. 7 and 8A-8C, data collected by a diagnosis/treatment device 152 is distributed to remote devices, such as a physician's computer, with the assistance of another device operated by the user, such as the user's mobile telephone or computer. Such data can be communicated in other ways. FIGS. 10A-10C provide three different examples of transmitting data to a remote device.

In FIG. 10A, the diagnosis/treatment device 152 is configured to wirelessly transmit data using a suitable short-range wireless protocol, such as Bluetooth, ZigBee, or Wi-Fi, to one or more wireless access points (WAPs) 230. In some embodiments, the WAP 230 that receives the data is a WAP owned or operated by the user. In other embodiments, the WAP 230 is a publicly-accessible WAP that is hosted by a business (e.g., coffee shop, bookstore, etc.) or other organization (e.g., public library). In still other embodiments, the WAP 230 can be a private WAP to which the diagnosis/treatment device user is granted access. In the latter case, the WAP owner or operator can grant such access to one or more such device users, for example, in return for a fee or discounted Internet access.

Irrespective of what entity owns/operates the WAP 230, the WAP is connected to a physical network 232, such as the wired Internet. Therefore, the WAP 230 can be used as a gateway to the Internet and to any remote devices, such as computers 234 and 236, that are connected to the Internet.

In FIG. 10B, the diagnosis/treatment device 152 is also configured to wirelessly transmit data using a suitable short-range wireless protocol such as Bluetooth, ZigBee, or Wi-Fi. In the embodiment shown in FIG. 10B, however, the diagnosis/treatment device 152 does not directly communicate with a WAP 230. Instead, the diagnosis/treatment device 152 wirelessly broadcasts data to one or more nodes 238, which then relay the data to the WAP 230 for transmission to remote computers via a physical network 232. In some embodiments, the nodes 238 comprise stationary nodes (e.g., desktop computers). In other embodiments, the nodes 238 comprise mobile nodes, such as mobile telephones, PDAs, wireless game consoles, laptop computers, and the like. Irrespective of the nature of the nodes 238, the owner/operator of each node grants the user the privilege of forwarding data on the user's behalf. Because the determination as to which nodes will forward the data may occur dynamically based upon network connectivity, the communication scheme shown in FIG. 10B may be described as a partial ad-hoc communication scheme.

Turning to FIG. 10C, illustrated is a fully ad-hoc communication scheme in which the no physical network (i.e., infrastructure) is used to transmit data. Instead, data transmitted by the diagnosis/treatment device 152 is relayed by a plurality of different nodes 240 to the intended recipient device, in this case a mobile telephone 242.

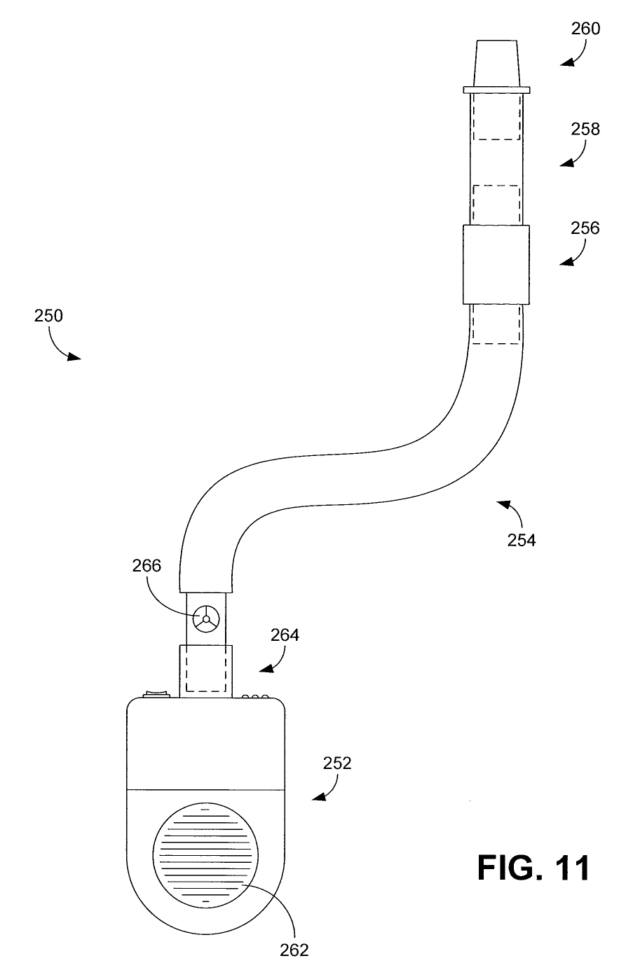

FIG. 11 illustrates a further embodiment of a device 250 for diagnosing and treating a respiratory condition, such as asthma. In the embodiment of FIG. 11, the device 250 takes the form of a respirator device that can be used to supply purified air to the user. The device 250 includes a respirator unit 252, a first hose section 254 that extends from the respirator unit, a medicine delivery unit 256 connected to the first hose section, a second hose section 258 that extends from the medicine delivery unit, and a mouthpiece 260 that is connected to the second hose section.

The respirator unit 252 comprises an internal fan (not shown) that draws air through an inlet 262 and forces the air through an internal filter (not shown) that filters the air by removing particulate matter from the air. The filtered or “purified” air is then expelled from the respirator unit 252 through an outlet 264 to which the first hose section 254 is connected. The purified air then travels through the first hose section 254 to the medicine delivery unit 256. Like the devices 10 and 152 described above, the medicine delivery unit 256 includes an internal medicine container and medicine delivery device (not shown) that can be used to administer medicine to the user. When medicine is delivered into the device flow path during user inhalation, the medicine can travel along with the purified air through the second hose section 258, into the mouthpiece 260, and into the user's respiratory system. The hose sections 254 and 258 may therefore be considered to comprise a drug delivery tube as is present in the device 10 shown in FIG. 1. As indicated in FIG. 11, the device 250 further comprises a pressure relief valve 266 that can exhaust air from the device when the user exhales into the mouthpiece 260. In some embodiments, the pressure relief valve 266 is provided adjacent the respiratory unit 252.

In addition to providing purified air to a user, the device 250 can further diagnose and treat a respiratory condition. Specifically, in similar manner to that described in the foregoing, pressures observed during user exhalations can be measured to determine the volume of the exhalations and make a judgement at to respiratory system health. In some embodiments, the pressures are measured using an internal differential pressure sensor provided within the medication delivery unit 256 (not shown). As with the device 11, the device 250 can estimate FEC₁ and VC from those pressures and determine the correct dosage of medication to administer, if any. In some embodiments, the estimation is performed by a controller provided within the medicine delivery unit 256 that also controls actuation of the unit's internal medicine delivery device. Once the correct dosage is determined, that dosage is administered to the patient using the medicine delivery unit 256.

Various modifications can be made to the above described methods and apparatuses. For example, the diagnosis/treatment device can include a override option that enables the user to obtain medication even if no testing is performed (e.g., in emergency situations) or if the results of the testing are negative. Furthermore, in embodiments in which the diagnosis/treatment device comprises a transceiver (e.g., transceiver 166 in FIG. 7), the transceiver can comprise a long-range wireless transceiver similar to that used in a mobile (e.g., cellular) telephone so that reliance on another computing device such as a separate mobile telephone or computer to transmit collected data is unnecessary. In such cases, treatment determinations can also be made remotely relative to data communicated from the diagnosis/treatment device. For instance, whether a patient is having an asthma attack and whether to administer medication and in what quantity can be determined by a remote computer that acts in similar manner to the control device described in relation to FIGS. 6-8. Moreover, global positioning system (GPS) hardware can be added to the diagnosis/treatment device such that the location of the device, and therefore its user, can be determined, for example when an emergency condition is detected. In some embodiments, the location of the user can be indicated in an emergency alert provided to the user's guardian, physician, or emergency medical service.

1. A respiratory condition device comprising:
a tube that defines a flow path for medicine that is to be delivered to a user respiratory system;
a pressure sensor configured to detect pressure changes within the tube; and
a medicine delivery device configured to eject medicine into the tube when a pressure drop is detected by the pressure sensor.

2. The respiratory condition device of claim 1, further comprising a mouthpiece in fluid communication with the tube.

3. The respiratory condition device of claim 1, wherein the pressure sensor comprises a silicon-based pressure sensor.

4. The respiratory condition device of claim 1, wherein the medicine delivery device comprises a droplet ejection device.

5. The respiratory condition device of claim 1, further comprising a medicine container that holds medicine to be delivered by the medicine delivery device.

6. The respiratory condition device of claim 1, further comprising an internal power source that provides power to the pressure sensor and the medicine delivery device.

7. The respiratory condition device of claim 1, further comprising an internal fan that generates airflow within the tube.

8. The respiratory condition device of claim 1, wherein the pressure sensor is further configured to detect a pressure increase indicative of the user exhaling into the tube and to measure a rate of flow of the user exhalation.

9. The respiratory condition device of claim 8, further comprising a controller configured to diagnose respiratory conditions from information collected by the pressure sensor.

10. The respiratory condition device of claim 9, wherein the respiratory conditions include a current or imminent asthma attack.

11. The respiratory condition device of claim 9, wherein the controller is further configured to control the medicine delivery device to eject medicine when a respiratory condition is diagnosed.

12. The respiratory condition device of claim 1, further comprising a data cable connector with which the respiratory condition device can communicate with a separate device.

13. The respiratory condition device of claim 1, further comprising a wireless transceiver with which the respiratory condition device can communicate with a separate device.

14. The respiratory condition device of claim 1, further comprising a user interface including a display and an input button.

15. A method for diagnosing a respiratory condition, the method comprising:
measuring pressures within a tube of a portable diagnosis device into which a user exhales;
estimating flow rates from the measured pressures;
determining the state of the user's respiratory system based on the flow rates; and
diagnosing a respiratory condition based upon the determined state.

16. The method of claim 15, wherein measuring pressures comprises measuring pressures within the tube of an independent handheld device.

17. The method of claim 15, wherein measuring pressures comprises measuring differential pressures with a silicon-based pressure sensor provided within the portable diagnosis device.

18. The method of claim 15, wherein estimating flow rates comprises estimating the flow rates using Ohm's law as adapted for fluid flow.

19. The method of claim 15, wherein determining the state comprises calculating a ratio between the user's forced expiratory volume in one second and the user's vital capacity.

20. The method of claim 15, wherein diagnosing a respiratory condition comprises determining that the user is having or is about to have an asthma attack if the ratio is below 75%.

21. The method of claim 20, further comprising providing an indication of the asthma attack to the user.

22. The method of claim 15, wherein the estimating, determining, and diagnosing are all performed by the portable diagnosis device and further comprising the portable diagnosis device transmitting an alert to a separate device.

23. The method of claim 15, wherein the estimating is performed by the portable diagnosis device and the determining and diagnosing are performed by a separate device to which the flow rates are transmitted from the portable diagnosis device.

24. A method for treating a respiratory condition, the method comprising:
a treatment device determining a dosage of medication to administer to a user;
the treatment device detecting inhalation of the user from a medicine delivery tube of the treatment device;
the treatment device controlling a medicine delivery device of the treatment device to output medicine into the delivery tube; and
the treatment device operating a fan of the treatment device to generate an air flow within the delivery tube to carry medicine output by the medicine delivery device to the user.

25. The method of claim 15, wherein determining a dosage comprises determining a dosage based upon a ratio between the user's forced expiratory volume in one second and the user's vital capacity.

26. The method of claim 15, wherein determining a dosage comprises receiving with the treatment device a dosage transmitted to the treatment device by a remote physician.

27. A method for diagnosing and treating a respiratory condition, the method comprising:
measuring pressures within a tube of a diagnosis/treatment device into which a user exhales;
estimating on the diagnosis/treatment device flow rates from the measured pressures;
determining on the diagnosis/treatment device the state of the user's respiratory system based on the flow rates;
diagnosing on the diagnosis/treatment device a respiratory condition based upon the determined state;
determining on the diagnosis/treatment device a dosage of medication to administer to a user;
detecting with the diagnosis/treatment device inhalation of the user from the tube of the diagnosis/treatment device;
controlling a medicine delivery device of the diagnosis/treatment device to output medicine into the delivery tube; and
operating a fan of the diagnosis/treatment device to generate an air flow within the delivery tube to carry medicine output by the medicine delivery device to the user.

28. A system for alerting as to a respiratory condition, the system comprising:
a diagnosis device configured to measure pressures within a tube of the diagnosis device into which a user exhales, to estimate flow rates from the measured pressures, to determine the state of the user's respiratory system based on the flow rates, and, if it is determined that the user is experiencing or about to experience a respiratory condition, to transmit notification to a separate device; and
a separate device in communication with the diagnosis device configured to receive notifications from the diagnosis device concerning determined respiratory conditions and transmit related alerts to remote devices via a network.

29. The system of claim 28, wherein the diagnosis device is further configured to administer medication to the respiratory system of user.

30. The system of claim 28, wherein the separate device comprises a mobile telephone.

31. The system of claim 28, wherein the separate device comprises a computer.

32. The system of claim 28, wherein the diagnosis device and the separate device communicate with each other via a data cable.

33. The method of claim 28, wherein the diagnosis device and the separate device wirelessly communicate with each other.

34. The method of claim 28, wherein the separate device is configured to transmit an alert as a telephone message, a text message, or an email message.

35. A method for alerting as to a respiratory condition, the method comprising:
measuring pressures within a tube of a diagnosis device into which a user exhales;
estimating flow rates from the measured pressures;
determining the state of the user's respiratory system based on the flow rates; and
if the user is determined to have a respiratory condition, transmitting an alert to a remote device using a separate device in electrical communication with the diagnosis device.

36. The method of claim 35, wherein the separate device comprises a mobile telephone.

37. The method of claim 35, wherein the separate device comprises a computer.

38. The method of claim 35, further comprising the diagnosis device and the separate device communicating with each other via a data cable.

39. The method of claim 35, further comprising the diagnosis device and the separate device wirelessly communicating with each other.

40. The method of claim 35, wherein transmitting an alert comprises the separate device transmitting a telephone message.

41. The method of claim 35, wherein transmitting an alert comprises the separate device transmitting a text message.

42. The method of claim 35, wherein transmitting an alert comprises the separate device transmitting an email message.

43. The method of claim 35, wherein the measuring, estimating, and determining are performed by the diagnosis device.

44. The method of claim 35, wherein the measuring is performed by the diagnosis device and the estimating and determining are performed by the separate device.
